(19) United States
(12) Patent Application Publication
(10) Pub. No.: US 2011/0009925 A1
(43) Pub. Date: Jan. 13, 2011

(54) TRANSCUTANEOUS RECEIVING ANTENNA DEVICE FOR IMPLANT

(75) Inventors: C. Roger Leigh, East Ryde (AU); Andrew L. Abela, Horsley Park (AU); Mark A. Von Huben, Waverton (AU); Andrian R. Cryer, Pymble (AU)

Correspondence Address: JOHN S. PRATT, ESQ. (Cochlear);KILPATRICK STOCKTON LLP, 1100 PEACHTREE STREET, SUITE 2800, ATLANTA, GA, 30309, US

(73) Assignee: COCHLEAR LIMITED, Lane Cove, NSW (AU)

(21) Appl. No.: 12/446,189

(22) PCT Filed: Oct. 15, 2007
(86) PCT No.: PCT/AU07/01561

§ 371 Date: Sep. 29, 2010

(30) Foreign Application Priority Data
Oct. 17, 2006 (AU) 2006905752

(51) Int. Cl. A61N 1/375; H01Q 7/00

(52) U.S. Cl. 607/60; 343/866

(57) ABSTRACT

An implantable antenna (16) for receiving and/or transmitting a signal is provided. The antenna (16) comprises a carrier (14) and an electrical conductor embedded in the carrier (14). The electrical conductor is formed from a foil.

### CROSS-REFERENCE TO RELATED APPLICATIONS

The present application is a National Stage application of PCT/AU2007/001561 entitled “Transcutaneous Receiving Antenna Device For Implant”, filed on Oct. 15, 2007, which claims priority from Australian Provisional Patent Application No. 2006905752, filed on Oct. 17, 2006, which are hereby incorporated by reference herein.

### BACKGROUND

1. Field of the Invention

The present invention relates to a transcutaneous receiving antenna device of a medical implant in a patient having an inductive coupling of electromagnetic energy and/or RF signals between an external antenna and the implanted antenna through the skin of the patient, as used for example in a hearing prosthesis or other implanted device.

2. Related Art

Certain types of active medical implants, such as cochlear prostheses, typically include an external component having an external coil antenna and an implanted component having an implanted coil antenna to form the transcutaneous link of the medical implant. The coils are arranged to provide an inductive coupling of RF signals and power therebetween through the skin of the patient.

The external component usually includes the transmitter coil antenna, a microphone, and a signal processor to receive, process and inductively transmit audio signals to the implanted component. The implanted component 1, as shown in prior art FIGS. 1-3, conventionally comprises the receiver coil antenna 6, an implant stimulator 2, and an implant transducer 3 to inductively receive, process and stimulate auditory nerve fibres to create the perception of sound in the brain. The implanted component 1 of the cochlear prosthesis is usually implanted in or near the mastoid region of the skull 5 behind the ear of the patient, and the transducer element 3 is implanted within the scala tympani of the cochlear in close proximity to the ganglion cells to thereby stimulate the auditory nerve fibres of a patient suffering sensorineural hearing loss. The external component is typically detachably secured in proximity to the implanted component by a magnetic attraction between a magnet resident in the external component and a magnet 8 resident in the implanted component.

For example, U.S. Pat. No. 6,327,504 describes the general principles of a common transcutaneous link and a method to reduce eddy currents arising between the coils by having circuitry and magnetic elements perpendicular to the plane of the main coil of the implanted component. Also, in U.S. Pat. No. 6,430,444, another transcutaneous energy transfer device is described that uses multiple coils to control the energy transfer in the transcutaneous link.

However, in these and other known medical implant devices, the implant coils are prone to fatigue typically concentrated at the joint where the coil is electrically connected to the stimulator. The implanted component 1 typically comprises a rigid stimulator casing 2, which contains the electrical assembly and a semi-rigid wire coil 6 in a semi-flexible carrier 4. The implant coils are formed from round wire, which is typically formed to shape manually and held in shape by moulding in a silicone elastomer to form the semi-flexible carrier 4.

The curvature of the head varies from patient to patient. Children and particularly infants generally have a much tighter curvature than average, and the implanted coil can put pressure on the skin at the part of the coil distal relative the stimulator where it is important for the coil to conform the head. In extreme cases, the coil may extrude through the skin.

United States Patent Application Publication No. 2004/0133065 describes a semi-implantable hearing aid having an external transmitter coil and an implanted receiver coil. The magnet 8, as shown in FIG. 3, is positioned in the carrier 4 at the centre of the circular coil 6, i.e. in an axial orientation relative to the circular coil, in the implanted component 1. The magnet in the implanted component is used to attract a similar magnet in the external component of the hearing aid to hold the external coil in place over the implanted coil. Being in the centre of the coil the magnets reduce the effective area of the coil, and the magnet blocks flux at the centre of the coils. Also the magnet material is not biocompatible and must be hermetically enclosed, typically in titanium.

In some implanted devices, for example in ceramic cased cochlear implants (for example MedE1, MXM devices), the magnet resides in the stimulator 2 together with the implanted coil. Since the stimulator case in the ceramic implants is non-conducting, the coil may function well within the ceramic case of the stimulator 2. In non-ceramic medical implants, typically titanium implants, the material of the housing of the stimulator 2 is conductive and the placement of the magnet and the implanted coil is usually required to be outside the conductive stimulator case.

There is a need for an implanted receiving antenna device suitable for an implanted device which improves on the structures of the medical implants discussed above.

### SUMMARY

In one aspect of the present invention there is provided an implantable antenna for receiving and/or transmitting a signal, the antenna comprising a carrier, and an electrical conductor embedded in the carrier, the electrical conductor being formed from a foil.

In another aspect of the present invention there is provided an implantable antenna for receiving and/or transmitting a signal, the antenna comprising a carrier and an electrical conductor embedded in the carrier, the electrical conductor being formed into a shape other than circular.

In another aspect of the present invention there is provided an implantable device comprising an antenna for receiving and/or transmitting a signal, said antenna comprising a carrier, and an electrical conductor embedded in the carrier, the electrical conductor being formed from a foil; wherein the conductor is formed by machining of the foil and welding any overlaid connections; wherein the conductor is formed by punching from the foil; wherein the conductor includes concentric elements; wherein said carrier is formed of an inner material and an outer material surrounding said inner material, wherein said outer material has a higher durometer than said inner material; wherein the electrical conductor being formed into a shape other than circular; wherein the conductor is shaped so as to modify the operative mechanical properties of the conductor; wherein the antenna is adapted to be connected at one end to an implanted device, wherein the antenna is relatively more flexible distal from the connection end than adjacent to the connection end; and wherein said carrier is formed of an inner material and an outer material surrounding said inner material, wherein said outer material has a higher durometer than said inner material.

In another aspect of present invention there is provided an implantable device comprising a stimulator and a separate carrier, the carrier including an electrical conductor embedded in the carrier so as to form an antenna for an inductive transcutaneous link, wherein a magnetic element is located in the stimulator outside the antenna so as to operatively allow the retention of a corresponding external antenna having an associated magnetic element. In another aspect of the present invention there is provided a device, including a second electrical conductor and a complementary magnetic element, adapted for use with an implantable device comprising a stimulator and a separate carrier, the carrier including an electrical conductor embedded in the carrier so as to form an antenna for an inductive transcutaneous link, wherein a magnetic element is located in the stimulator outside the antenna so as to operatively allow the retention of a corresponding external antenna having an associated magnetic element; and wherein the magnetic element is arranged to have a magnetic field with a specific geometry, so that operatively, a device with a complementary geometry is retained external to the body substantially in a desired orientation and position relative to the electrical conductor.

### BRIEF DESCRIPTION OF THE DRAWINGS

Preferred embodiments of the invention are described below in conjunction with the accompanying drawings, in which:

FIG. 1 is a simplified side elevation view of an implanted receiving antenna device of a known medical implant in situ;

FIG. 2 is a simplified side elevation view of an implanted receiving antenna device of a known medical implant;

FIG. 3 is a cross-sectional view taken along line A-A of FIG. 2 of an implanted receiving antenna device of a known medical implant;

FIG. 4 is a simplified side elevation view of an implanted receiving antenna device in accordance with an embodiment of the invention;

FIG. 5 is a simplified side elevation view of an implanted receiving antenna device in accordance with an embodiment of the invention;

FIG. 6A-B show plan views of implanted antennas in accordance with embodiments of the invention;

FIG. 7A-B show plan views of implanted antennas in accordance with embodiments of the invention;

FIG. 8A-H show cross-sectional views taken along line C-C of FIG. 6A of different embodiments of implanted antennas;

FIG. 9 is a cross-sectional view taken along line B-B of FIG. 5 of an implanted receiving antenna device in accordance with an embodiment of the invention.

FIG. 10 is a similar view to FIG. 9 showing an external antenna;

FIG. 11 is a plan view illustrating a possible connection arrangement between turns;

FIG. 12 is a plan view of a stamped antenna design;

FIG. 13 is a perspective view illustrating the folded antenna of FIG. 12;

FIG. 14 is a perspective view of an embodiment of an antenna; and

FIG. 15 is a perspective view of an alternative embodiment of an antenna.

### DESCRIPTION OF PREFERRED EMBODIMENT

The present invention will be described with reference to various specific implementations. However, it will be appreciated that these are intended to be illustrative rather than limiting.

The present invention is applicable to any implantable inductively linked system. It may be used for data only, for power only, or both power and data. It may be applied to hearing devices, for example hearing aids, cochlear implants, middle ear implants, brain stem implants, and in general to devices which provide mechanical or electrical stimulation or both. It may also be applied to other implanted or implantable devices, for example pacers and defibrillators, implantable neural stimulators or sensors, drug pumps, or any other situation where an inductive link is used to deliver power or data or both.

Some proposed implanted systems have a separate functional device, and implanted power supply. In such arrangements, the antenna for receiving power from the external device may be connected to the functional device, with power transferred for storage to the power supply, or the antenna may be associated with the power supply, which then supplies power to the functional device. The present invention is applicable to any of these alternatives. It may also be applied for power transfer between the functional device and the power supply.

FIG. 4 shows a side elevation view of an implanted receiving antenna device 10 in situ in accordance with an embodiment of the invention. The implanted receiving antenna device 10 is discussed herein with an implementation of a cochlear prosthesis; however, it will be appreciated that the device may be used in any implanted device where power and/or data are inductively transferred. The implanted receiving antenna device 10 includes a stimulator housing 12 and a flexible carrier 14 that conforms to the skull 5 of a patient. FIG. 5 shows how a transducer element 3 in simplified block form is provided and may be arranged to communicate, e.g. electrically connected or the like, with the electrical components (not shown) of the stimulator 12 to stimulate auditory nerve fibres to create the perception of sound in the brain in the patient. Such transducer elements are well known in the art.

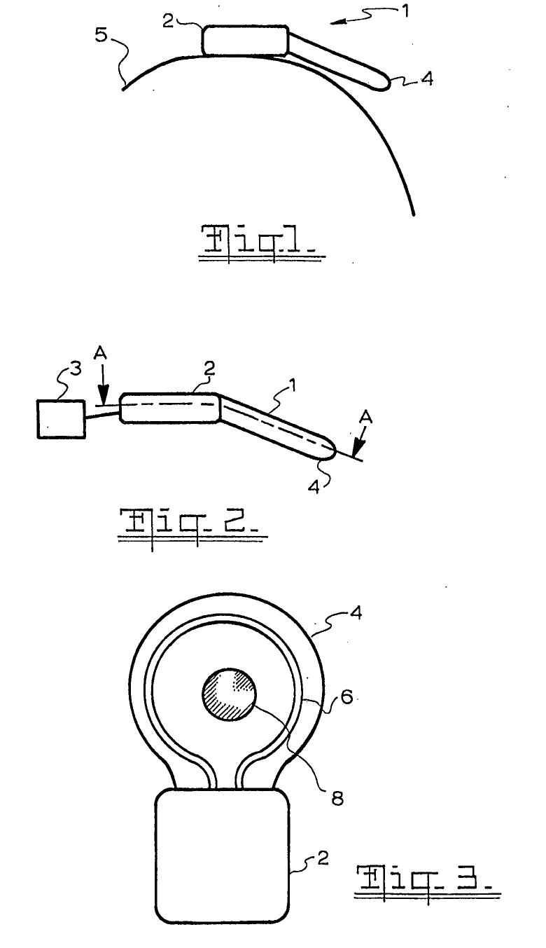

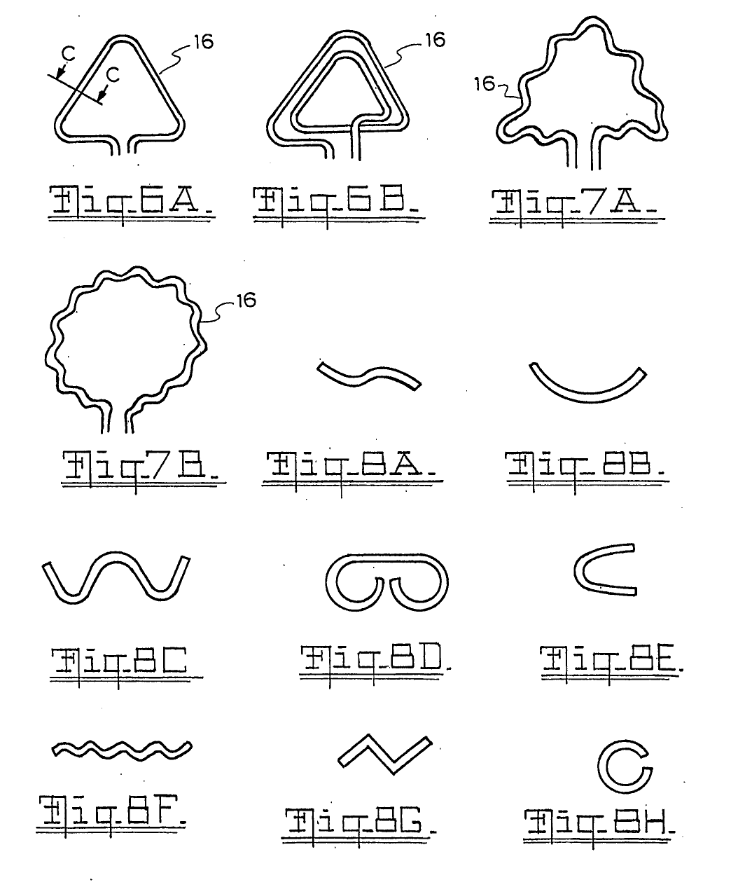

The implant antenna such as that in a cochlear implant transfers power and signals via an RF transcutaneous link. Conventionally, implant antennas are circular or round. In the design of the antenna shown here with reference to FIG. 6A, the implanted antenna 16 may be triangular in shape. The area of the antenna may be similar to existing implant antennas, which is approximately 700 mm². This shape provides the antenna with improved resistance to fatigue. In a conventional coil antenna, stresses causing fatigue are concentrated at the joint with the stimulator because this is the narrowest point of the antenna as well as the interface with the rigid stimulator. With the shape shown in FIG. 6A the stresses will not be concentrated at this point but will tend to be distributed over the antenna. It also provides the antenna with improved flexibility in the parts distal to the stimulator where it is important for the antenna to conform to the head. The stimulator housing 12 of the implant comprises a housing that is rigid typically being formed of titanium. The stimulator housing 12 contains the electrical assembly (not shown). Electrically connected to the electrical assembly via a feedthrough arrangement is the semi-flexible antenna 16. Traditionally, the flexibility of the antenna is limited by the need to contain a metal conductor and to have a minimum robustness, and the angle between the stimulator and antenna is chosen to be a reasonable fit to the average head, which is, for example, typically no less than 15 degrees. However, some heads, particularly those of infants, are of much tighter curvature than 15 degrees, being 45 degrees or more in a newborn child. It is important that in these circumstances where the curvature of the head is less than 15 degrees, the distal end, relative to the end of the antenna connected to the stimulator, conforms to the head of the patient and does not exert pressure on the skin of the patient which may cause discomfort to the patient. In these situations, in traditional applications, the pressure is typically greatest in the region where the mismatch between antenna and skull is greatest, i.e. distal to the stimulator. This pressure is minimised by the flexibility of the antenna. Hence improving the flexibility in this region is particularly beneficial. The triangular antenna has increasing flexibility with distance from the stimulator and conforms to tighter radius skulls than achievable previously, as shown in comparing FIG. 1 of the prior art with FIG. 4 of an embodiment of the present invention.

Traditionally implant antennas are formed from round wire. This wire is typically formed to shape manually and held in shape by moulding in a silicone elastomer. The implanted antenna 16 as shown in FIG. 6A of an embodiment of the invention is fabricated by punching or cutting from a sheet of conductive material, such as for example 0.1 mm thick platinum foil which is thinner than traditional wire used in similar applications. The foil may be any suitable biocompatible metal, for example gold, platinum, iridium and alloys thereof. Existing antennas typically consist of a twisted bundle of fine wires in which the overall diameter is 0.8 to 1 mm. With this fabrication process, the handling issues previously associated with gold wire which is soft and prone to damage during fabrication are overcome. This makes fabrication easier than traditional methods, as the techniques for fabrication from foil lend generally themselves well to automation.

A preferred process for forming the foil antenna starts with a sheet of foil for example of platinum foil 0.1 mm thick. The foil is adhered to a suitable carrier substrate using an adhesive. The carrier may be, for example, a block of a nylon or other similar polymer material. The carrier with the foil affixed is then processed by a CNC micro-machining system, which makes the required cuts in the foil according to a programmed shape. The substrate is then exposed to a solvent for the adhesive, releasing the cut foil components from the substrate. Prior to over moulding, any necessary connections or interconnections can be performed by welding, for example. Depending upon the design, it may be necessary to insert a layer of an insulating, biocompatible material to space any overlayed parts of the antenna from each other. This may be, for example, a silicone material, or any suitable biocompatible material.

Of course, the foil could be formed using any other suitable process, for example punching, which would be particularly viable in high volume production.

The conductor material is thinner than before, hence the overall thickness of the antenna may be significantly reduced and the flexibility increased. The antenna is embedded in the flexible carrier 14, which is typically formed using a mould but may, alternatively, be sprayed or dipped. The flexible carrier may be any flexible insulating biocompatible material to contain the implanted antenna. Materials for the flexible carrier include, for example, polyurethane, silicone or proprietary blends such as santoprene. In practice silicone elastomer is generally preferred for its proven long term biocompatibility. Complex shapes may be fabricated easily, such as the triangular shapes shown in FIGS. 6A and 6B. Also other much more complex shapes may be fabricated for example those shown in FIGS. 7A and 7B which provide some lateral flexibility. It will be appreciated that any number of shapes may be considered to achieve the desired functionality and give the advantage of flexibility where required and robustness where required. For example, the transcutaneous antenna may take the form other than triangular or circular, such as arch shape, square, oval, and the like. FIG. 14 illustrates a preferred antenna shape.

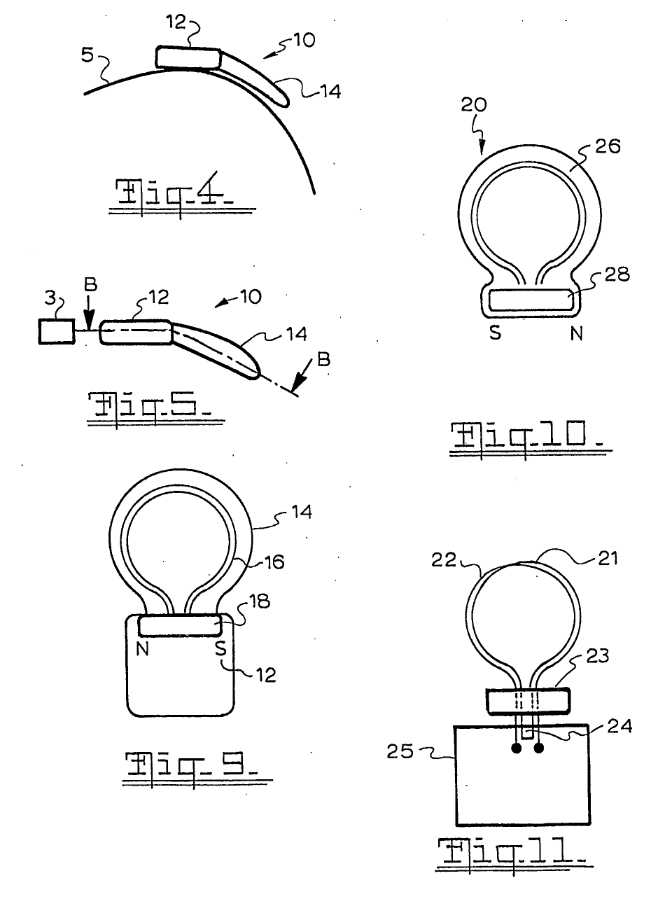

The traditionally circular implant antenna has a magnet positioned at its centre as shown in FIG. 3. The magnet in the transcutaneous antenna device attracts a similar magnet in the external antenna to hold the external antenna in place over the implant antenna. Being in the centre of the antenna the magnet reduces the effective area of the antenna and the magnet blocks flux at the centre of the antenna. Typically the magnet is 11 mm diameter so reduces the effective antenna area by about 14%. Also the magnet material is not biocompatible so must be hermetically enclosed, typically in titanium. As shown in FIG. 9, the magnet is positioned inside the stimulator 12 which is a hermetic enclosure for the implant electronics. Fabrication is simplified by requiring only a single hermetic enclosure and the effective antenna area is increased. Additionally, the area of the antenna may be configured in a smaller more compact arrangement for the same efficiency. If the antenna area is reduced by 14% but there is no magnet the effective area and hence efficiency will be largely unchanged. In traditional circular antenna and magnet arrangements, the magnets are at the centre of circular implant and external antennas with the north and south poles perpendicular to the plane of the antenna, to allow the external and implanted antennas to self align. In this embodiment, where the magnet is located in the stimulator, a different method of self alignment is required. This is achieved by having the poles of the magnet in the plane of the antenna, as shown in FIG. 9. The poles of the external magnet are reversed relative to those of the magnet residing in the implant antenna. The external antenna/magnet configuration 20 is shown in FIG. 10. Magnet 28 mirrors magnet 18, in use. As a consequence, the correct alignment is achieved without the use of a central magnet. Antenna 26 is ideally of comparable area to implanted antenna 16. Although the antennas are shown as circular, it will be appreciated that any desired shape as discussed herein could be used.

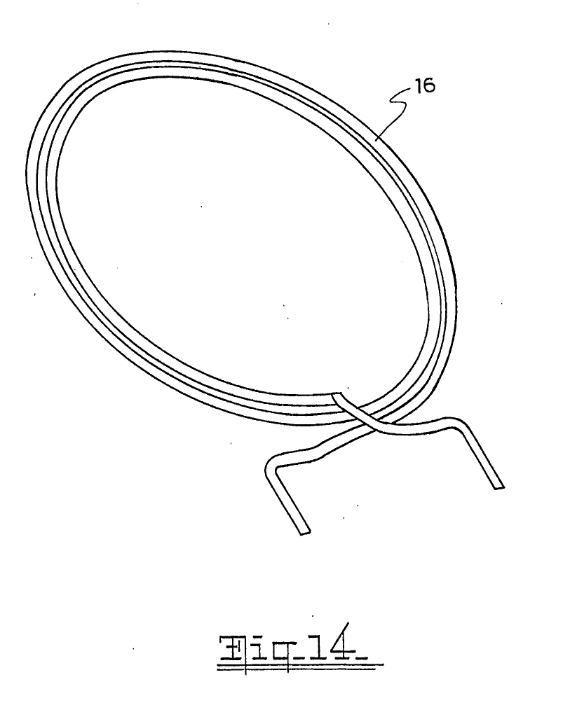

The configuration of the antenna may include any number of turns, for example multiples of the desired shape or shapes, and either one within the other, one on top of the other such that each turn is coaxially aligned or one beside the other, coaxially aligned, however having different dimensions. Additionally, multiple turn antennas are typically more efficient than single turn antennas. Traditional multiple turn antennas are created by making more than one turn of the same piece of wire. A preferred embodiment includes multiple punched turns arranged one on top of the other. This maximises the area of individual turns without making the overall size larger. To form the multiple layers into one continuous conductor, i.e. a multiple turn antenna, the ends of each layer may be connected to the feedthrough, which connects through to the electrical assembly in the stimulator. The interconnections between layers may be made on the PCB of the electrical assembly. FIG. 11 illustrates such an arrangement. Turns 21, 22 pass through feedthrough 23 into the electrical assembly 25. The connection 24 between the turns made be made using the PCB or a similar arrangement.

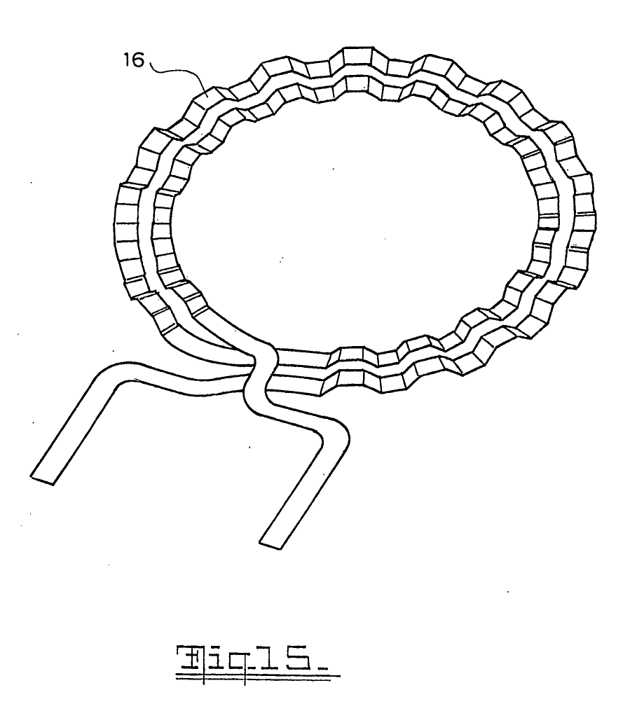

An alternative arrangement is to stamp out a shape which can be folded into a multiple turn antenna. One example is illustrated in FIGS. 12 and 13. The stamped shape 30 is folded along lines shown as 31, 32, and 33. This produces a two turn antenna arrangement, as shown in FIG. 13. An insulating material, for example in the form of a sheet, may be placed between each layer. It is preferred that the spacing is minimised, preferably to less than 1 mm, to keep the overall thickness minimised. This arrangement could be readily extended to more turns. The fold points are preferably reinforced to prevent fatigue. This could be achieved by applying extra over mould material at these points, or making the conductor thicker at these points, or some combination of these techniques.

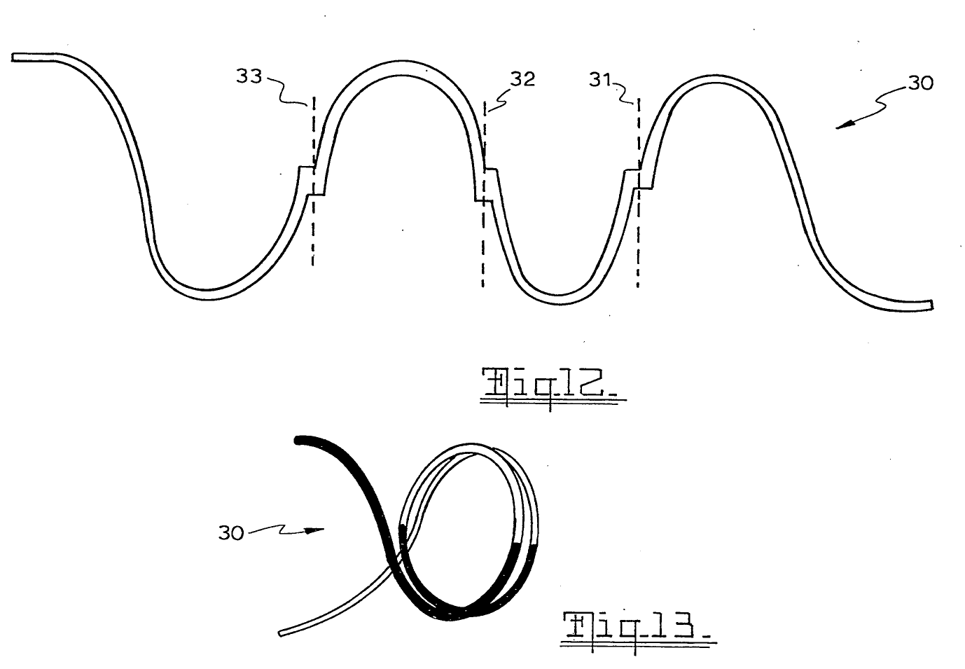

The conductor material of the antenna may be a foil, and the conductor material of the antenna may be of any biocompatible conductor e.g. platinum, iridium, stainless steel, titanium or suitable alloys. Alternatively, a non-biocompatible conductor could be coated with a robust biocompatible material, e.g. copper may be coated with stainless steel, using filled drawn tube. Of course, the conductor material may be thinner or thicker than 0.1 mm. The conductor material, for example foil, of the antenna may be laminated with insulation between multiple turns to improve the circuit Q factor. The conductor material of the antenna, for example the foil itself, may be shaped, as shown in FIGS. 8A-H to modify the mechanical properties, for example the flexibility and fatigue resistance, and/or electrical properties. The shaping of the foil may be applied over the whole antenna or only some sections. This shaping may enhance the electrical properties of the antenna by increasing surface area which will improve the RF coupling of the antenna. The shaping may also improve the mechanical properties, in particular the resistance to flexing or fatigue in the plane of the antenna. The preferred shaping of the antenna may also increase surface area, enhance in plane fatigue resistance, be easy to shape as part of the punching process and be stackable if more than one turn is used. A selection of shapes which could be tried are shown in FIGS. 8A-H. In particular, in order to appropriately provide strain relief, it may be advantageous to use an antenna which has curves or bends extending both in the plane of the antenna, and perpendicular to the plane. This may be in the form of the arrangements shown in FIGS. 7A, 7B, 8F or 15 for example, where the curved or bent portions are not confined to the plane of the antenna.

In an embodiment, to further optimise flexibility and robustness the antenna moulding may be fabricated of silicone elastomers having two or more degrees of hardness. The inner volume of the antenna may be moulded from a soft silicone, for example 30 durometer. This may be overmoulded using a higher durometer silicone, for example 60 durometer. The inner soft silicone may provide the antenna with flexibility, while the outer hard silicone may provide the antenna with robustness against damage. Typical damage that may be prevented includes, for example, nicks to the antenna which may provide access points for body fluid which if exposed to conducting material of the antenna may cause implant failure.

In an embodiment, two implant options may be supplied, one optimised to fit the shape of the left side of the skull and one for the right side of the skull. Additionally, the device of FIG. 9 may be arranged to include any number of magnets, and rather than a bar shape magnet as shown, the magnet may include two or more round (or other shape) magnets.

It will be understood that the foregoing description of a number of embodiments of the present invention is for the purposes of illustration only, and that the various structural and operational features and relationships disclosed herein are susceptible to a number of modifications and changes none of which entails any departure from the scope of the invention as defined in the appended claims.

1. An implantable antenna for receiving and/or transmitting a signal, said antenna comprising a carrier, and an electrical conductor embedded in the carrier, the electrical conductor being formed from a foil.

2. The antenna according to claim 1, wherein the conductor is formed by machining of the foil and welding any overlaid connections.

3. The antenna according to claim 1, wherein the conductor is formed by punching from the foil.

4. The antenna according to claim 1, wherein the conductor includes concentric elements.

5. The antenna according to claim 1, wherein said carrier is formed of an inner material and an outer material surrounding said inner material, wherein said outer material has a higher durometer than said inner material.

6. An implantable antenna for receiving and/or transmitting a signal, said antenna comprising a carrier, and an electrical conductor embedded in the carrier, the electrical conductor being formed into a shape other than circular.

7. The antenna according to claim 6, wherein the conductor is shaped so as to modify the operative mechanical properties of the conductor.

8. The antenna according to claim 7, wherein the antenna is adapted to be connected at one end to an implanted device, wherein the antenna is relatively more flexible distal from the connection end than adjacent to the connection end.

9. The antenna according to claim 6, wherein said carrier is formed of an inner material and an outer material surrounding said inner material, wherein said outer material has a higher durometer than said inner material.

10. An implantable device comprising an antenna for receiving and/or transmitting a signal, said antenna comprising a carrier, and an electrical conductor embedded in the carrier, the electrical conductor being formed from a foil;
wherein the conductor is formed by machining of the foil and welding any overlaid connections;
wherein the conductor is formed by punching from the foil;
wherein the conductor includes concentric elements;
wherein said carrier is formed of an inner material and an outer material surrounding said inner material, wherein said outer material has a higher durometer than said inner material;
wherein the electrical conductor being formed into a shape other than circular;
wherein the conductor is shaped so as to modify the operative mechanical properties of the conductor;
wherein the antenna is adapted to be connected at one end to an implanted device, wherein the antenna is relatively more flexible distal from the connection end than adjacent to the connection; and
wherein said carrier is formed of an inner material and an outer material surrounding said inner material, wherein said outer material has a higher durometer than said inner material.

11. An implantable device comprising a stimulator and a separate carrier, the carrier including an electrical conductor embedded in the carrier so as to form an antenna for an inductive transcutaneous link, wherein a magnetic element is located in the stimulator outside the antenna so as to operatively allow the retention of a corresponding external antenna having an associated magnetic element.

12. The implantable device according to claim 11, wherein the magnetic element is arranged to have a magnetic field with a specific geometry, so that operatively, a device with a complementary geometry is retained external to the body substantially in a desired orientation and position relative to the electrical conductor.

13. A device, including a second electrical conductor and a complementary magnetic element, adapted for use with an implantable device comprising a stimulator and a separate carrier, the carrier including an electrical conductor embedded in the carrier so as to form an antenna for an inductive transcutaneous link,
wherein a magnetic element is located in the stimulator outside the antenna so as to operatively allow the retention of a corresponding external antenna having an associated magnetic element; and
wherein the magnetic element is arranged to have a magnetic field with a specific geometry, so that operatively, a device with a complementary geometry is retained external to the body substantially in a desired orientation and position relative to the electrical conductor.
